(19) United States
(12) Patent Application Publication
(10) Pub. No.: US 2004/0156928 A1
(43) Pub. Date: Aug. 12, 2004

(54) Adhesive label with fluidifying agents for natural airway secretions

(76) Inventors: Gunter Cordes, Leichlingen (DE); Ulrike Vollmer, Langenfeld (DE)

Correspondence Address: HARNESS, DICKEY & PIERCE, P.L.C., P.O.BOX 8910, RESTON, VA, 20195, US

(21) Appl. No.: 10/450,899

(22) PCT Filed: Dec. 18, 2001
(86) PCT No.: PCT/EP01/14945

(30) Foreign Application Priority Data
Dec. 19, 2000 (DE) 10063378.1

(51) Int. Cl. A61K 35/78; A61K 9/70

(52) U.S. Cl. 424/742; 424/449

(57) ABSTRACT

Label for the outerwear for thinning of airway secretions.

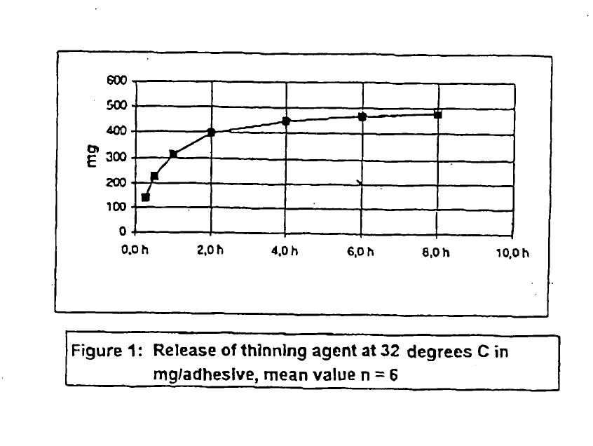

Colds are a widespread ailment. They are characterized by the disagreeable result of congestion of the upper airway by a considerably increased amount of endogenous secretions, caused by the activity of viruses and/or bacteria. Thus, an immediate alleviation for the infected body exists, when draining of the secretions is facilitated.

It has long been known that the ingredients of plants that are rich with ether oils are suitable for this facilitation of draining. Such ether oils can be included as tea or in capsules (for example, Gelomyrtol®, Pohl-Boskamp), but often also cause irritation of the stomach and gall bladder. Therefore, often the topical use in the form of salves are used, which contain ether oils and which are to be applied by the patient on the chest, so that the substance penetrates the skin and enters the body via the airways. Corresponding preparations are Wick VapoRub® (Wick Pharma), Bronchoforten® (Plantorgan), and Pinimenthol® (Spitzner). The duration of the substance release is limited to approximately one to two hours. A further problem of the salve application lies in the contamination of the hands with the slimy, skin-irritating ether oils. In order to avoid eye contact, it is essential that the patient washes his hands after use.

In order to avoid these disadvantages, carrier systems of non-woven material or fabric have been developed, which absorb the substance and enable a simpler application. These are either placed closed to the body (DE 4204222, DE 4007275, DE 3911617, DE 3823395) or adhered to the skin (DE 3540515) and then release the ether oils over a long time period on the skin side as well as into the atmosphere. In other developments, the material reservoir is enclosed between two films that are permeable to the material (DE 3902981, DE 3216609).

The use of salves as well as the above-described systems bring the substances that are suitable for inhalation also in contact with the skin. The irritation of the skin by various ether oils (rosemary oil, turpentine oil, camphor) is known and is used for treatment of rheumatoid ailments. With the treatment of cold related illnesses with ether oils, this accompanying effect is not desired, however. But with common salve preparations and the above-described reservoir systems, this cannot be avoided.

The present invention represents a system, with which the noted disadvantages can be avoided. Thus, the present invention operates as a label, which contains a thinning agent that, with the help of endogenous body head, enters into the natural body openings of the upper airways and there leads to a liquefying of the secretions caused by the cold.

The object on which the present invention is based is thereby resolved by a label, which is characterized in that it can be adhered to clothing worn close to the body and which has a thinning agent, which is released from the adhered label to the surrounding environment of the clothing wearer and enters into the natural body openings of the upper airways and can liquefy accumulated airway secretions caused by the cold.

The label of the present invention can be characterized by an adhesive layer and/or a layer for sticky adhesion, a removable protective layer for the adhesive layer or the sticky adhesive layer and a non-woven material containing a thinning agent, in particular, a mixture of two or more ether oils as the thinning agent.

In addition, the label of the present invention can be characterized by a thinning agent that can release an initial dose at the beginning of the use and thereafter, a maintenance dose of the thinning agent over a longer time period for liquefying of the accumulated airway secretions caused by the cold, whereby the release rate in milligrams of thinning agent/non-woven layer surface/hour of the initial dose is greater than that of the maintenance dose.

Furthermore, the label of the present invention can be characterized in that the thinning agent can release an initial dose of 100 to 300 mg/hour, preferably of approximately 150 mg/hour, over the first two hours after beginning the use, and thereafter, over at least six hours, a maintenance dose of 10 to 30 mg/hour, preferably of approximately 15 mg/hour, for liquefying accumulated airway secretions caused by a cold.

In addition, the label of the present invention can be characterized in that the adhesive layer can be attained exclusively from the monomers methyl-acrylate, 2-ethyl-hexl-acrylate, and acrylic acid by radical copolymerization with the additional of a cross-linking agent.

Further, the label of the present invention can be characterized in that the adhesive layer can be attained with aluminum acetyl acetonate as the cross-linking agent, in particular, in an amount of 0.04 to 1% (with reference to a weight of all monomers).

Additionally, the label of the present invention can be characterized in that the adhesive layer and the non-woven material layer have been connected to one another when in a wet state and thereafter have been dried.

In addition, the label according to the present invention can be characterized by a synthetic spun mat as the non-woven material layer (carrier mat), in particular, with a coating weight per unit area of 70 to 130 g/m².

Furthermore, the label of the present invention can be characterized by polyester, rayon, Trevira, Dralon, or Modal as the material of the synthetic spun mat.

In addition, the label according to the present invention can be characterized by a fleecy-appearing and/or white or colored non-woven mat layer.

Additionally, the label of the present invention can be characterized by a mixture of ether oils as the thinning agent, wherein the mixture includes an ether oil, which, with a temperature of the clothing worn close to the body, in particular, in the range of 30 to 34° C., does not have a low viscosity or is solid, and the mixture also includes an ether oil, which is fluid with the temperature of the clothing worn close to the body, wherein the mixture of the oils with temperatures of the clothing worn close to the body likewise is fluid, without the need for other assisting materials.

Further, the label according to the present invention can be characterized by a mixture of ether oils of a plant base as the thinning agent, wherein the ether oils are serviceable for secretolysis of airway secretions.

In addition, the label of the present invention can be characterized by a mixture of ether oils from plant components, whose contents or primary contents are selected from a group of terpenes, preferably from a group of monoterpenes, in particular, from the group consisting of 1,8-cineol=eucalyptol, camphor, camphene, menthol, aterpinol, thymol, pinene, and limonene.

Furthermore, the label of the present invention can be characterized by eucalyptus oil as the thinning agent or as one of its components.

The label of the present invention can be further characterized in that the thinning agent contains a mixture of eucalyptus oil and camphor as the ether oil, or comprises preferably a weight ratio of eucalyptus oil:camphor of approximately 3:1.

The label according to the present invention can also be characterized by a size of 20 to 200 cm² and preferably 30 to 60 cm².

In addition, the label of the present invention can be characterized in that one label or multiple labels that are sufficient for an acute cold are found in one package, which preferably is gas impermeable.

The penetration of medications in the natural openings of the body surfaces of the airways is determined substantially by the physical-chemical qualities of the substance. In this regard, the vapor pressure and the temperature of ebullition or the volatility of a substance play a role. Here, it was surprisingly found that the eutectic and self-liquefying mixture of the liquid eucalyptus oil and the solid, crystallized camphor, in a combination of approximately 3:1, enters excellently into the body openings of the airways from the label and in addition, leads to a liquefying of the secretions there. It is no longer necessary to use ether oils in addition co the label. No further vehicle is necessary, such as the turpentine oil, alcohol, Vaseline, etc., used in other topical salve formulations, in order to transport the contents to the airways. It is known that turpentine oil strengthens the liquefying effect; indeed, it was also determined that the migration of the turpentine oil is so incense that is seeps through the packaging means, in which case, the stability and security bases are also detrimentally affected.

Also, by application of the label on the clothing of the body infected by the cold, a contact of the otherwise irrigating ether oils with all mucous membranes (eyes, stomach) and other body surfaces (skin) is avoided, and in spite of that, the airways blocked with secretions are reached. These secretions comprise mucous, which regulate the secretions via disulfide bridges to the protein polymers. It has been shown now that the exclusive separation of the disulfide bridges, for example, by acetyl-cysteine (Fluimucil® products, Zambon) leads not so certainly to the desired facilitation, such as hydrating of the mucous, which is accomplished by eucalyptus oil as well as by guaifenesine. As a supplement, the camphor works here, which leads to a cold irritation on the mucous membranes and therewith, counteracts the inflammation-indicating heat (hyperemia).

It is important that when ether oils are released in a non-diluted form for inhalation, the release of the oils takes place non-abruptly, because then the ether oils can be released in too concentrated of a form and can lead to a two-phase reverse effect, which can be undesirable. The ether oils should be diluted so highly that the odor is only slightly discernible (Boyd and Sheppard, 1970). This is optimally achieved by the combination of the label (application of the oil in an absorption mat) and the selection of the body temperature of 30-34° C., as the evaporation behavior with in vitro-measurement makes clear. No additional supplements for diluting, such as ethanol, Vaseline, and so on are required. By means of the control by the non-woven material and temperature, emissions in two phases or speeds can be achieved, specifically, a higher rate in the first two hours (initial dose with approximately 150 mg oil/hour) and a lower, so-called maintenance dose (with approximately 15 mg oil/hour) after two hours, which stops after at least six hours. Thus, the label is particularly well suited for use overnight on pajamas.

It is important with the manufacturing of such a label with this type of effective liquefying system that the thinning agent does not engage and liquefy the polymers on the label. It was discovered that the use of a completely specialized acrylate-copolymer in connection with a defined amount of cross-linking agent for antagonizing eventual liquefying of the label leads to a stable product, without using other protective materials, such as inhibiting films, aluminum damping, or the like, makes possible an optimal adhesion on various surfaces of the clothing (natural fibers, such as wool or cotton, as well as synthetic materials, such as polyester, polyamides, etc.). and is removable without residue. A particularly good connection of the adhesion matrix with the absorption mat is achieved, in that in a wet, that is, a solution-retaining state, the connection between both layers is created and is subsequently dried. Then, also no debonding or pulling of filaments can take place by the addition of liquefying ether oils.

As the material for absorbing the ether oils, an absorptive, somewhat thicker non-woven material is suitable. The best are non-woven materials (“nonwovens”) with a coating weight per unit area of at least 70 to 130 g/m², which are manufactured, for example, from types of polyester or rayon. As the protective film for the adhered side of the label, a siliconized polyester film, for example. Hostaphan RN 100 from Diafoil, Noechst, Germany, easy/easy, that is known to the practitioner can be used, which should not be too thin (at least 36 μm layer thickness, preferably 100 μm layer thickness), so that the label can be used well in practice from 30 to 200 cm², preferably, from 30 to 60 cm².

The following embodiments serve for a more detailed explanation of the invention:

### EXAMPLE 1

In order to manufacture 100 m² label rolls, one adds 0.051 kg aluminum acetyl acetonate to 28.858 kg of a 35% (m/m) solution of an acrylate-adhesive (Durotak 87-2852. National Starch and Chemical B.V., NL-Zutphen). By stirring every half hour, the solution is homogenized and subsequently spread with a coating knife onto a siliconized, 100 μm thick polyester film (FL 2000 100μ 1-S, Rexam Release B.V., NL-Apeldoorn) in a wet layer thickness of 400 μm. Before drying, it is covered with Paramoll N260/100 (polyester non-woven material of the Fa. Lohmann, D-Andernach) and subsequently dried (15 minutes at 70° C.). The homogenous laminate formed thereby is made into individual labels of 60 cm² by cutting. Directly before packaging in composite packaging material-sealed pouches, the labels are applied by means of a spray nozzle with 500 mg of the ether oil mixture of eucalyptus oil:camphor of 3:1. One obtains a label, which contains an adhesive part of 102 g/m² and 20% eucalyptus oil as well as 6.7% camphor.

### EXAMPLE 2

In order to make 100 m² rolls of labels, one adds 0.051 kg aluminum acetyl acetonate to 28.858 kg of a 35% (m/m) solution of an acrylate-adhesive (Durotak 87-2852, National Starch and Chemical B.V., NL-Zutphen) . By stirring every half hour, the solution is homogenized and subsequently spread with a coating knife onto a siliconized, 100 μm thick polyester film (FL 2000 100μ 1-S, Rexam Release B.V., NL-Apeldoorn) in a wet layer thickness of 400 μm. Before drying, it is covered with TWE-non-woven material 100 (rayon non-woven material of Fa. TWE, D-Emsstaetten) and subsequently dried (15 minutes at 70° C.). The homogeneous laminate that is thereby made is made into individual labels of 59 cm² by stamping. Directly before packaging in composite packaging material-sealed pouches, the labels are applied by means of a spray nozzle with 500 mg of the ether oil mixture of eucalyptus oil:camphor of 3:1. One obtains a label, which contains an adhesive part of 102 g/m² and 20% eucalyptus oil as well as 6.7% camphor.

1. A label that can be adhered to clothing worn close to a body and that has a thinning a which is released from the adhered label to the surrounding environment of the clothing wearer and enter into the natural body openings of the upper airways and which can liquefy accumulated airway secretions caused by a cold, wherein the thinning agent comprises a self-liquefying mixture of eucalyptus oil and camphor.

2. The label according to claim 1, characterized by an adhesive layer and/or a layer for sticky adhesion, a removable protective layer for the adhesive layer or sticky adhesion layer and a non-woven material layer with a content of thinning agent, in particular, a mixture of two or more ether oils as thinning agents.

3. The label according to claim 1 or 2, characterized by a thinning agent, which to begin a use, can release an initial dose and thereafter over a longer time period, an release a maintenance dose of thinning agent for liquefying the accumulated airway secretions caused by a cold, wherein a release rate in milligrams of thinning agent/non-woven material layer surface/hour of the initial dose is greater than a release rate of the maintenance dose.

4. The label according to claim 3, characterized in that the thinning agent can release the use of an initial dose of 100 to 300 mg/hour, preferably of approximately 150 mg/hour, over the first two hours after beginning the use, and thereafter, over at least six hours, a maintenance dose of 10 to 30 mg/hour, preferably of approximately 15 mg/hour, for liquefying accumulated airway secretions caused by a cold.

5. The label according to one of claim 2 to 4, characterized in that the adhesive layer can be attained exclusively from the monomers methyl-acrylate, 2-ethyl-hexl-acrylate, and acrylic acid by radical copolymerization with the additional of a cross-linking agent.

6. The label according to claim 5, characterized in that the adhesive layer can be attained with aluminum acetyl acetonate as the cross-linking agent, in particular, in an amount of 0.04 to 1% (with reference to a weight of all monomers).

7. The label according to claim 5, characterized in that the adhesive layer and the non-woven material layer have been connected to one another when in a wet state and thereafter have been dried.

8. The label according to one of the preceding claims, characterized by synthetic spun mat as the non-woven material layer (carrier mat) in particular, with a coating weight per unit area of 70 to 130 g/m².

9. The label according to claim 8, characterized by polyester, rayon, Trevira, Dralon, or Modal as a material of the synthetic spun mat.

10. The label according to one of claims 8 or 9, characterized by a fleecy-appearing and/or white or colored non-woven mat layer.

15. The label according to one of claims 2 through 14, characterized in that the thinning agent comprises a mixture of eucalyptus oil and camphor as the ether oils in a weight ratio of eucalyptus oil:camphor of approximately 3:1.

16. The label according to one of the preceding claims, characterized by a size of 20 to 200 cm² and preferably 30 to 60 cm².

17. The label according to one of the preceding claims, characterized in that one label or multiple labels that are sufficient for an acute cold are found in one package. which preferably is gas impermeable.
